(19) United States
(12) Patent Application Publication
(10) Pub. No.: US 2011/0111998 A1
(43) Pub. Date: May 12, 2011

(54) MODIFICATION OF PARTICULATE-STABILISED FLUID-FLUID INTERFACES

(76) Inventors: Harry Javier Barraza, Wirral (GB); Sejong Kim, Greensboro, NC (US); Orlin Dimitrov Velev, Cary, NC (US)

(21) Appl. No.: 12/920,356

(22) PCT Filed: Feb. 26, 2009
(86) PCT No.: PCT/EP2009/052293

§ 371 Date: Jan. 6, 2011

(30) Foreign Application Priority Data
Mar. 14, 2008 (EP) 08152783.0

(51) Int. Cl. A61K 8/03; A61Q 13/00; C09K 3/00

(52) U.S. Cl. 510/119; 512/2; 510/513; 510/159; 252/182.12

(57) ABSTRACT

The invention provides a composition comprising at least two immiscible fluid phases separated by a fluid-fluid interface, in which the interface is stabilised by an assembly of biopolymeric microparticles adsorbed at the interface, characterised in that the properties of the interface are modified via the association of at least one functional group on the biopolymer for example hydroxypropyl methyl cellulose phthalate with at least one ligand for example eosin. This enables, for example, the production of coloured emulsions and in particular coloured foams and bubbles.

### FIELD OF THE INVENTION

The present invention relates to compositions comprising at least two immiscible fluid phases separated by a fluid-fluid interface, in which the fluid-fluid interface is stabilised by a solid particulate.

### BACKGROUND AND PRIOR ART

Fluid-fluid interfaces are ubiquitous in industrial and consumer products. For example, most personal care products available in the market involve emulsions, suspensions or dispersions of various immiscible fluid phases.

Foams occur as end products or during use of products in a wide range of areas including the detergent, food and cosmetic industries. They are mixtures of immiscible fluids in which a gas phase is dispersed as bubbles in the continuous phase of a liquid.

To prevent collapse of the foam, surfactants are usually added whose molecules cover the liquid/vapour interfaces. Also, certain small solid particles such as nanosilicas have been shown to exhibit some similarities with such molecules by adsorbing at interfaces and acting to stabilise droplets in emulsions and bubbles in foams.

In WO2007/068344 fibres are modified to impart surface active properties to them. The modified particles may be used for emulsion stabilisation. Modification may be done by coating the fibres with a hydrophobic material such as ethylcellulose or hydroxypropyl cellulose. The coating is deposited onto the fibre in a separate process step. The processes exemplified use ethyl cellulose and the coated fibre particles are separated and dried before they can be used for foam stabilisation. The particles onto which the polymer is coated are described as having a length of several tens of microns. Neither the fibre nor the deposited coating can be considered to be a small molecule or ligand.

WO2008/046732 describes frozen aerated products comprising surface active fibres of the type disclosed in WO2007/068344. The ethyl cellulose is typically prepared in acetone solution. As with the earlier patent, the process requires the pre-formation of the coated rods, and as before neither the coating nor the rod/fibre material can be considered to be a small molecule or ligand, as defined herein.

In recent years, much attention has been devoted to what have been termed smart or intelligent materials. Such materials have the capability to sense changes in their environment and respond to the changes in a pre-programmed and pronounced way. For example, smart polymers undergo fast and reversible changes in microstructure triggered by small changes of medium property (pH, temperature, ionic strength, presence of specific chemicals, light, electric or magnetic field. These microscopic changes of polymer microstructure may, for example, manifest themselves at the macroscopic level as a precipitate formation in a solution. The change is reversible. In this patent specification, the term “biopolymer” is used to describe smart polymers that are derived from natural (biological) sources. One such well known class of biopolymers are the enteric polymers that dissolve on change of pH and are capable of delaying release of a drug from an ingested capsule, coated with the enteric polymer, until after it has passed through the acid environment of a stomach. Another well known use of such polymers is the purification of biological materials (ligands) by the attachment of the ligand to the polymer as it precipitates on change of pH and the subsequent release of the ligand from the polymer after separation from the solvent.

One enteric polymer has been investigated for foam stabilisation. Drug Development and Industrial Pharmacy, 33:141-146, 2007 Vol. 33, No. 2, December 2006: pp. 1-16 Study of the Effect of Stirring on Foam Formation from Various Aqueous Acrylic Dispersions; describes the use of Eudragit type biopolymers to stabilise foams made by high speed stirring of aqueous solutions of the polymers and pH adjustment.

Coloured foams have been considered as a desirable product format for many years. A history of their development in relation to aerosol products is given in “coloured foams for children” in Spray technology and Marketing, March 2003, pages 49-53.

US 2006/0004110 describes compositions and methods for producing coloured bubbles. Several of the examples use acid dyes. The process to make the bubbles uses high temperature to dye glycerine, which is then incorporated into the composition. The glycerine is not a solid particulate stabilising system so it must be used with other adjuncts, which may stabilise the bubbles.

We have found that certain biopolymeric interfacial stabilisers are able to modify fluid-fluid interfaces by associating with small molecules such as dyes. This enables, for example, the production of coloured emulsions, and in particular coloured foams and bubbles. The invention is therefore especially applicable to product sectors where visual product appeal is an important aspect, such as cosmetics and personal care.

### SUMMARY OF THE INVENTION

The invention provides a composition comprising at least two immiscible fluid phases separated by a fluid-fluid interface, in which the interface is modified by microparticles of biopolymer adsorbed at the interface, characterised in that the microparticles are associated via at least one functional group on the biopolymer with at least one ligand.

The invention further provides a process for forming the composition comprising modified interfaces.

### DETAILED DESCRIPTION OF THE INVENTION

### Biopolymeric Microparticles

In the composition of the invention, the interface is stabilised by an assembly of biopolymeric microparticles adsorbed at the interface.

The microparticles may be anisotropic. Such microparticles will typically have an aspect ratio greater than 1 and are then preferably rods or fibres.

Suitable biopolymers used to form the microparticles have hydrophobic properties and possess surface functional groups with affinity to dyes or other small molecules (such as perfumes, proteins, and crosslinkers). Such molecules are referred to herein as ligands.

Examples of such biopolymers include hydrophobically substituted polysaccharides whose solubility is a function of pH and/or temperature and which form anisotropic microparticles as described above when precipitated from solution.

A preferred class of such biopolymers comprises cellulosic polymers with at least one ester- and/or ether-linked substituent, in which the parent cellulosic polymer has a degree of substitution of at least one hydrophobic substituent of at least 0.1. “Degree of substitution” refers to the average number of the three hydroxyls per saccharide repeat unit on the cellulose chain that have been substituted. “Hydrophobic substituents” may be any substituent that, if substituted to a high enough level or degree of substitution, can render the cellulosic polymer essentially aqueous insoluble. Examples of hydrophobic substituents include: ether-linked alkyl groups (such as methyl, ethyl, propyl and butyl), ester-linked alkyl groups (such as acetate, propionate and butyrate) and ether-linked and/or ester-linked aryl groups (such as phenyl, benzoate and phenylate).

More preferably, the cellulosic polymer as defined above is also at least partially ionisable and also includes at least one ionisable substituent, which may be either ether-linked or ester-linked. Examples of ether-linked ionisable substituents include: carboxylic acids (such as acetic acid, propionic acid, benzoic acid and salicylic acid), alkoxybenzoic acids (such as ethoxybenzoic acid and propoxybenzoic acid), the various isomers of alkoxyphthalic acid (such as ethoxyphthalic acid and ethoxyisophthalic acid), the various isomers of alkoxynicotinic acid (such as ethoxynicotinic acid), the various isomers of picolinic acid (such as ethoxypicolinic acid), thiocarboxylic acids (such as thioacetic acid), substituted phenoxy groups (such as hydroxyphenoxy), amines (such as aminoethoxy, diethylaminoethoxy and trimethylaminoethoxy), phosphates (such as phosphate ethoxy) and sulphonates (such as sulphonate ethoxy). Examples of ester linked ionisable substituents include: carboxylic acids (such as succinate, citrate, phthalate, terephthalate, isophthalate and trimellitate), the various isomers of pyridinedicarboxylic acid, thiocarboxylic acids (such as thiosuccinate), substituted phenoxy groups (such as amino salicylic acid), amines (such as natural or synthetic amino acids, such as alanine or phenylalanine), phosphates (such as acetyl phosphate) and sulphonates (such as acetyl sulphonate).

Specific examples of such preferred cellulosic polymers include: hydroxypropyl methyl cellulose acetate succinate, hydroxypropyl methyl cellulose succinate, hydroxypropyl cellulose acetate succinate, hydroxyethyl methyl cellulose succinate, hydroxyethyl cellulose acetate succinate, hydroxypropyl methyl cellulose phthalate, hydroxyethyl methyl cellulose acetate succinate, hydroxyethyl methyl cellulose acetate phthalate, carboxyethyl cellulose, carboxymethyl cellulose, carboxymethyl ethyl cellulose, cellulose acetate phthalate, methyl cellulose acetate phthalate, ethyl cellulose acetate phthalate, hydroxypropyl cellulose acetate phthalate, hydroxypropyl methyl cellulose acetate phthalate, hydroxypropyl cellulose acetate phthalate succinate, hydroxypropyl methyl cellulose acetate succinate phthalate, hydroxypropyl methyl cellulose succinate phthalate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate, and ethyl picolinic acid cellulose acetate.

Particularly preferred are cellulosic polymers that are aqueous insoluble in their nonionised state but aqueous soluble in their ionised state. A particular subclass of such polymers are the so-called “enteric” polymers, which are aqueous insoluble at pH 5.0 or less, but which become aqueous soluble at pH values above this threshold. Accordingly, these materials can form anisotropic microparticles (as described above) at pH 5.0 or less, which will dissolve or disrupt as solution pH increases.

Specific examples of such enteric polymers include, for example, hydroxypropyl methyl cellulose acetate succinate (HPMCAS), hydroxypropyl methyl cellulose phthalate (HPMCP), cellulose acetate phthalate (CAP), cellulose acetate trimellitate (CAT), and carboxymethyl ethyl cellulose (CMEC). In addition, non-enteric grades of such polymers, as well as closely related cellulosic polymers, may also be suitable due to the similarities in physical properties.

Mixtures of any of the above described materials may also be used, as can mixtures of different molecular weights of a particular material. The use of such mixtures enables the tuning of mechanical properties of the interface such as elasticity. This may be advantageous for producing foams of enhanced stability. The inclusion of high molecular weight hydroxypropyl methyl cellulose phthalate in such mixtures has been found to enhance foam stability. Examples of such mixtures include mixtures of this material with either (i) lower molecular weight hydroxypropyl methyl cellulose phthalate, or (ii) hydroxypropyl methyl cellulose acetate succinate; in which the weight ratio of high molecular weight hydroxypropyl methyl cellulose phthalate to (i) or (ii) is at least 1:1, more preferably at least 2:1, most preferably at least 3:1. By “high molecular weight” is meant at least 100,000 g/mol, more preferably 130,000 g/mol or more. By “lower molecular weight” is meant less than 95,000 g/mol, more preferably 85,000 g/mol or less.

### Ligand

In the composition of the invention, the properties of the interface are modified via the association of at least one functional group on the biopolymer with at least one ligand.

Suitable ligands have an affinity for surface functional groups on the biopolymer (such as the cellulose polymers which are described above).

Suitable ligands are able to modify optical and/or functional properties of the interface via their association with the biopolymer, and include small molecules such as dyes, perfumes, proteins, crosslinkers or the like. Such molecules are referred to herein as ligands. By small molecules we mean those having preferably a molecular weight of less than 500 Da, more preferably less than 350 Da. Ligands that have been found to bind particularly well to the functionalised biopolymers under the high shear conditions preferred to form the stabilised foams encompassed by the invention, comprise one or more aromatic rings. Among such compounds are aromatic perfumes, such as benzyl acetate.

This use of the expression ligand is a development of the definition of ligands in biochemistry published in 1992 by the joint commission on Biochemical Nomenclature [Arch. Biochem. Biophy., 1992 294 322-325.]: “If it is possible or convenient to regard part of a polyatomic molecular entity as central, then the atoms, groups or molecules bound to that part are called ligands”.

Examples of suitable ligands include acidic dyes. By “acidic dye” (or “acid dye”) is generally meant a coloured aromatic compound that has an overall negative charge in solution. Generally, acidic dyes have functional groups such as azo, triphenylmethane or anthraquinone that include acidic substituents such as hydroxyl, carboxyl or sulphonic groups.

A preferred class of ligand for use in the invention comprises those acidic dyes which exhibit a pH-dependent affinity for biopolymers such as the “enteric” polymers which are described above.

The use of these materials is preferred since the strong adsorption affinity of the dye for the biopolymer enables the production of modified interfaces (such as coloured foam) which are stable when set in conventional external fluid phases. Surprisingly such modified interfaces are also stable in the presence of surfactants, which is particularly advantageous when formulating products with a significant level of surfactant such as hair and body cleansers.

Examples of preferred acidic dyes are those materials which will protonate at pH 5.0 or less, i.e. those pH values at which the enteric polymer is aqueous insoluble and can form microparticles as described above.

Accordingly, preferred acidic dyes include weak acid groups such as hydroxyl and/or carboxyl groups in the dye structure.

In structural terms, a preferred class of acidic dyes comprises acidic xanthene dyes.

The class of xanthene dyes contains a xanthene nucleus, as shown below in formula (I), which is substituted at various positions. The xanthene dye class is covered by indices 45000 to 45999 in the Colour Index.

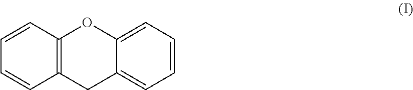

The acidic xanthene dyes preferred for use in the invention include hydroxyl and/or carboxyl substituent groups in the dye structure, more preferably hydroxyl and carboxyl substituent groups in the dye structure.

A particularly preferred subclass of the above described acidic xanthene dyes contains a fluorone nucleus, as shown below in formula (II), which is typically further substituted at various positions with substituents such as halogen.

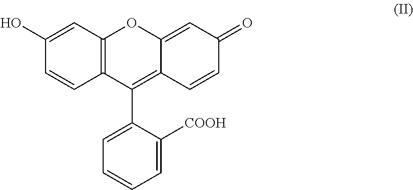

Specific examples of preferred acidic dyes are listed in the Table below. The Colour Index numbers (C.I.) are taken from the Colour Index International, 4th Edition Online, published by the Society of Dyers and Colourists in association with the American Association of Textile Chemists and Colorists.

| Chemical or other name(s) | C.I. | Colour |
|---|---|---|
| Acid Yellow 73; Uranine; disodium 2-(3-oxido-6-oxo- | 45350 | Yellow |
| xanthen-9-yl)benzoate | | |
| Solvent Yellow 94; Fluorescein; 2-(6-hydroxy-3-oxo- | 45350:1 | Yellow |
| (3H)-xanthen-9-yl)benzoic acid | | |
| Acid Orange 11; disodium 4′,5′-dibromo-3-oxospiro[2- | 45370 | Orange |
| benzofuran-1,9′-xanthene]-3′,6′-diolate | | |
| D&C Orange No. 5; Eosinic acid; 4′,5′-dibromo-3′,6′- | 45370:1 | Orange |
| dihydroxyspiro[2-benzofuran-3,9′-xanthene]-1-one | | |
| Acid Red 87; Eosin Y; disodium 2-(2,4,5,7- | 45380 | Red |
| tetrabromo-3-oxido-6-oxo-xanthen-9-yl)benzoate | | |
| Solvent Red 43; 2′,4′,5′,7′-tetrabromo-3′,6′- | 45380:2 | Red |
| dihydroxyspiro[2-benzofuran-3,9′-xanthene]-1-one | | |
| Solvent Orange 16; 3′,6′-dihydroxy-4′,5′- | 45396 | Orange |
| dinitrospiro[2-benzofuran-3,9′-xanthene]-1-one | | |
| Acid Red 91; Eosin B; 4′,5′-dibromo-3′,6′-dihydroxy- | 45400 | Red |
| 2′,7′-dinitrospiro[2-benzofuran-3,9′-xanthene]-1-one | | |
| Acid Red 98; Phloxine K; dipotassium 2′,4′,5′,7′- | 45405 | Red |
| tetrabromo-4,7-dichloro-3-oxospiro[2-benzofuran-1,9′- | | |
| xanthene]-3′,6′-diolate | | |
| Acid Red 92; Phloxine B; disodium 2′,4′,5′,7′- | 45410 | Red |
| tetrabromo-4,5,6,7-tetrachloro-3-oxospiro[2- | | |
| benzofuran-1,9′-xanthene]-3′,6′-diolate | | |
| Solvent Red 48; 2′,4′,5′,7′-tetrabromo-4,5,6,7- | 45410:1 | Red |
| tetrachloro-3′,6′-dihydroxyspiro[2-benzofuran-3,9′- | | |
| xanthene]-1-one | | |
| Acid Red 95; disodium 4′,5′-diiodo-3-oxospiro[2- | 45425 | Red |
| benzofuran-1,9′-xanthene]-3′,6′-diolate | | |
| Solvent Red 73; 3′,6′-dihydroxy-4′,5′-diiodospiro[2- | 45425:1 | Red |
| benzofuran-3,9′-xanthene]-1-one | | |
| Acid Red 51; Erythrosin B; disodium 2-(2,4,5,7- | 45430 | Red |
| tetraiodo-3-oxido-6-oxo-xanthen-9-yl)benzoate | | |
| Acid Red 94; Rose Bengal; disodium 2,3,4,5- | 45440 | Red |
| tetrachloro-6-(2,4,5,7-tetraiodo-3-oxido-6- | | |
| oxoxanthen-9-yl)benzoate | | |

Mixtures of any of the above described materials may also be used.

### Formation of Modified Interfaces

In a preferred process for forming modified interfaces according to the invention, biopolymeric microparticles are prepared by a precipitation process in which a solution of biopolymer is precipitated under conditions of high shear. Such high shear conditions for an aqueous non-viscous composition can suitably be created using a high shear mechanical mixing device, such as a rotor-stator type device, operating at rotational speeds ranging from between 7000 to 20000 rpm. Ultrasonic dispersers, homogenizers and other shear intensive apparatuses could also be used to prepare the biopolymeric microparticles.

Once the biopolymeric microparticles are created, they can be used to associate with a ligand (for example via the pH-dependent affinity mechanism which is described above for enteric polymers and certain acidic dyes). The associated polymer-ligand complex so formed can then be used in conjunction with lower shear, or frothing equipment to create modified fluid-fluid interfaces according to the invention.

In a particularly preferred process for forming modified interfaces according to the invention, a solution of enteric polymer at pH greater than 5.0 is precipitated by acidification of the solution under conditions of high shear and in the presence of an acidic dye which has a pH-dependent affinity for the enteric polymer, and which will protonate at pH 5.0 or less (such as the acidic xanthene dyes described above). The resulting mixture is then allowed to settle and a coloured foam is obtained, in which the air-liquid interface is stabilised by microparticles of enteric polymer in association with acidic dye.

Alternatively, or additionally the particles of enteric polymer can be precipitated in the presence of dispersed perfume and can bind to such perfume ligands in a similar manner.

The skilled worker will readily appreciate that any suitable ligand may become associated with any biopolymer that can be precipitated in its vicinity, especially under high shear conditions and that such a system has the ability to form the associated biopolymer and ligand to become preferentially located at the fluid-fluid interface. Thus, when dyes are used as ligands they can make intensely coloured stable foams while leaving no dye in the liquid beneath the foam. This movement of the ligand from the solution to the stabilised foam or emulsion is a particularly interesting effect that can obviously be exploited in a wide range of compositions and products.

### Product Form

Modified interfaces (such as coloured foams) according to the invention are stable in the presence of surfactants.

Accordingly the composition of the invention may advantageously be formulated as a home or personal care composition comprising one or more surfactants.

An example of a suitable product form is a personal wash composition such as a hair and/or body cleanser. Such a personal wash composition will comprise one or more cleansing surfactants which are cosmetically acceptable and suitable for topical application to the skin and/or hair.

Suitable cleansing surfactants, which may be used singly or in combination, are selected from anionic, amphoteric and zwitterionic surfactants, and mixtures thereof.

Examples of anionic surfactants are the alkyl sulphates, alkyl ether sulphates, alkaryl sulphonates, alkanoyl isethionates, alkyl succinates, alkyl sulphosuccinates, N-alkyl sarcosinates, alkyl phosphates, alkyl ether phosphates, alkyl ether carboxylates, and alpha-olefin sulphonates, especially their sodium, magnesium, ammonium and mono-, di- and triethanolamine salts. The alkyl and acyl groups generally contain from 8 to 18 carbon atoms and may be unsaturated. The alkyl ether sulphates, alkyl ether phosphates and alkyl ether carboxylates may contain from 1 to 10 ethylene oxide or propylene oxide units per molecule.

Typical anionic surfactants for use in personal wash compositions of the invention include sodium oleyl succinate, ammonium lauryl sulphosuccinate, ammonium lauryl sulphate, sodium dodecylbenzene sulphonate, triethanolamine dodecylbenzene sulphonate, sodium cocoyl isethionate, sodium lauryl isethionate and sodium N-lauryl sarcosinate. The most preferred anionic surfactants are sodium lauryl sulphate, triethanolamine monolauryl phosphate, sodium lauryl ether sulphate 1EO, 2EO and 3EO, ammonium lauryl sulphate and ammonium lauryl ether sulphate 1EO, 2EO and 3EO.

Examples of amphoteric and zwitterionic surfactants include alkyl amine oxides, alkyl betaines, alkyl amidopropyl betaines, alkyl sulphobetaines (sultaines), alkyl glycinates, alkyl carboxyglycinates, alkyl amphopropionates, alkylamphoglycinates, alkyl amidopropyl hydroxysultaines, acyl taurates and acyl glutamates, wherein the alkyl and acyl groups have from 8 to 19 carbon atoms. Typical amphoteric and zwitterionic surfactants for use in shampoos of the invention include lauryl amine oxide, cocodimethyl sulphopropyl betaine and preferably lauryl betaine, cocamidopropyl betaine and sodium cocamphopropionate.

The composition can also include co-surfactants, to help impart aesthetic, physical or cleansing properties to the composition. A preferred example of such a co-surfactant is a nonionic surfactant, which can be included in an amount ranging from 0% to about 5% by weight of the total composition.

For example, representative nonionic surfactants that can be included in personal wash compositions of the invention include condensation products of aliphatic (C8-C18) primary or secondary linear or branched chain alcohols or phenols with alkylene oxides, usually ethylene oxide and generally having from 6 to 30 ethylene oxide groups.

Other representative nonionics include mono- or di-alkyl alkanolamides. Examples include coco mono- or di-ethanolamide and coco mono-isopropanolamide.

Further nonionic surfactants which can be included in personal wash compositions of the invention are the alkyl polyglycosides (APGs). Typically, the APG is one which comprises an alkyl group connected (optionally via a bridging group) to a block of one or more glycosyl groups. Preferred APGs are defined by the following formula:

RO-(G)n

wherein R is a branched or straight chain alkyl group, which may be saturated or unsaturated, and G is a saccharide group. R may represent a mean alkyl chain length of from about C₅ to about C₂₀. Preferably R represents a mean alkyl chain length of from about C₈ to about C₁₂. Most preferably the value of R lies between about 9.5 and about 10.5. G may be selected from C₅ or C₆ monosaccharide residues, and is preferably a glucoside. G may be selected from the group comprising glucose, xylose, lactose, fructose, mannose and derivatives thereof. Preferably G is glucose. The degree of polymerisation, n, may have a value of from about 1 to about 10 or more. Preferably, the value of n lies in the range of from about 1.1 to about 2. Most preferably the value of n lies in the range of from about 1.3 to about 1.5.

Mixtures of any of the above-described materials may also be used.

The total amount of surfactant in personal wash compositions of the invention generally ranges from 0.1 to 50%, preferably from 5 to 30%, more preferably from 10% to 25% by total weight of surfactant based on the total weight of the composition.

Modified interfaces (such as coloured foams) according to the invention are also stable in the presence of external fluid phases, such as a surrounding fluid phase.

Accordingly, the composition of the invention may advantageously be formulated as a coloured foam, which is dispersed into a suspending base to form distinctive coloured air pockets or inclusions within the suspending base.

The suspending base will typically comprise one or more suspending agents for suspending the coloured foam in dispersed form in the suspending base or for modifying the viscosity of the suspending base.

Suitable suspending agents include organic polymeric materials, which may be of synthetic or natural origin. Specific examples of such materials include vinyl polymers (such as cross linked acrylic acid and crosslinked maleic anhydride-methyl vinyl ether copolymer), polymers with the CTFA name Carbomer, cellulose derivatives and modified cellulose polymers (such as methyl cellulose, ethyl cellulose, hydroxyethyl cellulose, hydroxypropyl methyl cellulose, nitrocellulose, sodium cellulose sulfate, sodium carboxymethyl cellulose, crystalline cellulose and cellulose powder), polyvinylpyrrolidone, polyvinyl alcohol, guar gum, hydroxypropyl guar gum, xanthan gum, arabia gum, tragacanth, galactan, carob gum, guar gum, karaya gum, carragheenin, pectin, agar, quince seed (Cydonia oblonga Mill.), starch (rice, corn, potato, wheat), algae colloids (algae extract), microbiological polymers (such as dextran, succinoglucan and pullulan), starch-based polymers (such as carboxymethyl starch and methylhydroxypropyl starch), alginic acid-based polymers (such as sodium alginate and alginic acid), propylene glycol esters, acrylate polymers (such as sodium polyacrylate, polyethylacrylate, polyacrylamide and polyethyleneimine).

Other suitable suspending agents include inorganic water soluble materials. Specific examples of such materials include bentonite, aluminium magnesium silicate, laponite, hectorite, and anhydrous silicic acid.

Other suitable suspending agents include crystalline fatty materials. Specific examples of such materials include ethylene glycol esters of fatty acids having from about 16 to about 22 carbon atoms (such as the ethylene glycol stearates, both mono and distearate), alkanolamides of fatty acids having from about 16 to about 22 carbon atoms (such as stearic monoethanolamide, stearic diethanolamide, stearic monoisopropanolamide and stearic monoethanolamide stearate), long chain esters of long chain fatty acids (such as stearyl stearate and cetyl palmitate), long chain esters of long chain alkanolamides (such as stearamide diethanolamide distearate and stearamide monoethanolamide stearate), glyceryl esters (such as glyceryl distearate, trihydroxystearin and tribehenin), N,N-dihydrocarbyl amido benzoic acid and soluble salts thereof (such as sodium and potassium salts), alkyl dimethyl amine oxides (such as stearyl dimethyl amine oxide), primary amines having a fatty alkyl moiety having at least about 16 carbon atoms (such as palmitamine and stearamine), secondary amines having two fatty alkyl moieties each having at least about 12 carbon atoms (such as dipalmitoylamine and di(hydrogenated tallow)amine) and di(hydrogenated tallow)phthalic acid amide.

Mixtures of any of the above-described materials may also be used.

The total amount of suspending agent in the suspending base at a concentration effective Such concentrations generally range from about 0.1% to about 10%, preferably from about 0.3% to about 5.0%, by total weight suspending agent based on the total weight of the composition.

Preferably the suspending base will also comprise other ingredients suitable for home or personal care compositions. For example, the suspending base may also comprise a surfactant such as those described above and in amounts as described above in relation to personal wash compositions.

### Optionals

Compositions of the invention may contain further ingredients as described below to enhance performance and/or consumer acceptability.

For example, skin or hair care actives may be included to provide skin or hair benefits in addition to cleansing. Examples of such benefits include hydration, nutrition, softness, protection and revitalisation.

Examples of typical skin or hair actives include glycerine, sorbitol, vitamins, botanical extracts, fruit extracts, sugar derivatives, alpha hydroxy acids, isopropyl myristate, UV filters, fatty acids and their esters, silicones, amino acids, hydrolysed proteins, cationic surfactants, essential oils, vegetable oils, mineral oils, sterols, cationic polymers, exfoliating agents and bactericides.

Other optional ingredients include fragrance, dyes and pigments, pH adjusting agents, pearlescers or opacifiers, viscosity modifiers and preservatives.

The above optional ingredients will generally be present individually in an amount ranging from 0 to 5% by weight individual ingredient based on the total weight of the composition.

The invention is further illustrated with reference to the following, non-limiting examples.

### EXAMPLES

### Example 1

### Formation of Coloured Foam

A solution of the enteric polymer hydroxypropylmethylcellulose phthalate (from Shin Etsu Chemical Co., HP 55 grade) was prepared by mixing 10 g of the material in 70 ml of deionised water, followed by addition of 21 ml of sodium hydroxide solution 1 N. This solution was stirred slowly for 12 hours to obtain a homogeneous clear solution. After this, the total volume was adjusted to 100 ml by adding deionised water.

10 ml of the above solution was then mixed with 0.1 ml of dye solution (1% w/v, Erythrosin B, C.I. 45430), and poured at slow speed into a running food blender containing 140 ml hydrochloric acid solution (1 N).

As the enteric polymer hits the acid solution the polymer molecules become less soluble and start interacting to form a suspension of particles. Under continuous shear (approximately 15000 rpm), the particles become substantially smaller until they reach the micron size range. At the same time, the dye becomes protonated and interacts with the enteric polymer.

After 60 seconds of the blending process, the whole contents were transferred into a 250 ml graduated cylinder. Minutes later, two distinct phases could be observed: a lower, transparent liquid phase; and an upper, pink coloured foam phase. The final pH of the transparent liquid phase was around 3.4.

The results demonstrate that the air-liquid interface of the foam is stabilised by microparticles of the enteric polymer in association with the dye, since the colour is confined to the foam.

### Example 2

### Coloured Foam Properties as a Function of pH

A range of four coloured foams (Samples A to D) were prepared using the methodology described in example 1 and using the same amounts and concentrations of hydroxypropylmethylcellulose phthalate and Erythrosin B dye, but with slight variations in the hydrochloric solution concentration so as to generate a range of final pH conditions in the liquid environment. The window of final liquid pHs was 3.3 to 4.6.

In all cases, a coloured foam phase was formed in equilibrium with a liquid phase. While the colour of the foam was similar in all experiments (a light pink), the liquid phase below the foam changed from completely transparent at lower pH values to hazy and slightly red at higher pHs. Table 1 below summarizes the observed behaviour.

TABLE 1

| | Sample A | Sample B | Sample C | Sample D |
|---|---|---|---|---|
| Amount of dye | 0.1 ml of 0.1% w/v Erythrosin B | | | |
| Final liquid | 4.6 | 4.4 | 4.1 | 3.3 |
| pH | | | | |
| Appearance of | Slightly | Slightly | Transparent | Transparent |
| liquid phase | red and | red and | (no colour | (no colour |
| | opaque | opaque | trace) | trace) |

This demonstrates that the affinity of the dye for the enteric polymer is pH-dependent, since at the higher pH values (Samples A and B), although a coloured foam is observed, the dye is not exclusively confined to the foam.

### Example 3

### Coloured Foam Properties as a Function of Dye Concentration

A range of four coloured foams were prepared using the methodology described in example 1 and using the same pH conditions and amount and concentration of hydroxypropylmethylcellulose phthalate, but with slight variations in dye concentration so as to generate a range of foams with different colour intensities (Erythrocin B, (0.1% w/v): 0.3 ml; 0.6 ml; 2.0 ml; and 4.0 ml).

In all four cases, a coloured foam in equilibrium with a transparent liquid phase was observed. As the amount of dye added increased, so too increased the intensity of the colour in the foam: changing from a light pink to a deep, bright red.

A methodology was developed to measure the colour intensity of the optically modified interfaces using of a UV-Vis spectrometer with an Integrating Sphere attachment (Jasco, ISV model). The absorption range measurement was set between 400 and 700 nm, and the dye-absorbing region (450-580 nm) was used to follow the intensity of absorption with the amount of dye. The absorption peak intensity increased with increasing amounts of dye, and levelled off when the amount of dye solution used approaches 1 ml.

From this data it is possible to conclude that there is a saturation value for the system, above which there is no further change in the optical properties of the interface.

It was noted that even at higher concentrations of dye there was no change in dye distribution between the foam and the liquid. Even when the amount of dye was 4 times the maximum level required to colour the interface (i.e. 4 ml), no dye migrated to the liquid phase. This demonstrates the strength of the affinity of the dye for the enteric polymer at the pH conditions used.

### Example 4

### Coloured Foam Properties in the Presence of Surfactant

A range of four coloured foams were prepared using the methodology described in example 1. For three of the foams, a constant amount of surfactant (0.05% w/v) was added to the acidic aqueous phase prior to the preparation of the foam, in order to test the influence of surfactant presence. Three different surfactant types were tested: sodium dodecyl sulfate (SDS); cetyltrimethyl ammonium bromide (CTAB); and polyoxyethylene (20) sorbitan monolaurate (Tween 20). Table 2 below summarizes the main observations.

TABLE 2

| | Normalized Foam Volume (%) | | | | |
|---|---|---|---|---|---|
| | time | | | | |
| Ingredient | t = 0 h | t = 25 h | t = 50 h | t = 75 h | T = 240 h |
| Hydroxypropyl | 80 | 45 | 42 | 40 | 38 |
| methylcellulose | | | | | |
| phthalate (HP) | | | | | |
| alone | | | | | |
| (HP) + SDS | 245 | 35 | 35 | 35 | 35 |
| (HP) + CTAB | 80 | 35 | 32 | 32 | 32 |
| (HP) + Tween 20 | 110 | 52 | 42 | 42 | 35 |

The data shows that the initial normalized foam volumes measured for coloured foams prepared in the presence of surfactants are substantially higher than foam volumes formed with HP alone. However, as time progresses the foam volumes approach equilibrium values that are close to the equilibrium volume values for HP-stabilized foams alone. This demonstrates that the stability of coloured foams according to the invention is not significantly affected despite the presence of various types of surfactant.

### Example 5

### Coloured Foam Formation with Different Enteric Polymers and Dyes

A range of enteric polymers were evaluated with a range of dyes for coloured foam formation and quality.

Coloured foams were generated as follows: 2.0 g of HCl (1N) was added to 276.4 g of deionised water to give a solution pH around 2.3. In a separate container, 1.2 g of dye solution (1% w/v) and 20.2 g of enteric polymer solution were thoroughly mixed. The aqueous phase was set in a beaker with a rotor-stator, high shear mixer (Silverson L4RT) at 10000 rpm. Very slowly, the dye/enteric polymer solution was added to the aqueous phase, and at the same time between 1.5 and 4.0 ml of HCl (1N) was added to set the final liquid pH in the range of 2.8 to 4.0. Coloured foam formed instantly after 2 to 5 min shearing was stopped. The results are shown in Table 4 below.

TABLE 4

| | | | | |
|---|---|---|---|---|
| Dye | Foam | Liquid | pH | Foam quality |

HP-55 (Standard hydroxypropylmethylcellulose, from Shin-Etsu)
Molecular weight: 84,000 g/mol
Critical pH (soluble/insoluble transition): 5.5

| | | | | |
|---|---|---|---|---|
| Erythrosin B | colour | clear | 3.7 | Good |
| (C.I 45430) | | | | |
| Eosin Y | colour | clear | 3.0 | Good |
| (C.I. 45380) | | | | |
| Eosin B | colour | clear | 3.3 | Good |
| (C.I. 45400) | | | | |
| Fluorescein | colour | clear | 3.6 | Good |
| (C.I. 45350:1) | | | | |

HP-55S (High molecular weight hydroxypropylmethylcellulose,
from Shin-Etsu)
Molecular weight: 132,000 g/mol
Critical pH (soluble/insoluble transition): 5.5

| | | | | |
|---|---|---|---|---|
| Erythrosin B | colour | clear | 3.8 | Good |
| Eosin Y | colour | clear | 3.2 | Good |
| Eosin B | colour | clear | 3.4 | Good |
| Fluorescein | colour | clear | 2.9 | Good |

AS-HF(hydroxypropylmethylcellulose acetate succinate,
from Shin-Etsu)
Molecular weight: 18,000 g/mol
Critical pH (soluble/insoluble transition): 6.8

| | | | | |
|---|---|---|---|---|
| Erythrosin B | colour | clear | 3.1 | Good |
| Eosin Y | colour | clear | 3.2 | Good |
| Eosin B | colour | clear | 3.5 | Good |
| Fluorescein | colour | clear | 3.2 | Good |

### Example 6

Further testing was conducted to study the stabilization properties of different enteric polymers for a single dye type (Eosin B). Mixtures of HP-55; HP-55S and AS-HF in different ratios were prepared and foams produced according to the methodology described above in Example 5.

Enhanced foam stability over an extended period of time (24 h) was observed for the mixtures shown below in Table 5.

TABLE 5

| Weight ratio | | Liquid | Initial | Stability |
|---|---|---|---|---|
| in mixture | Dye | pH | foam quality | after 24 h |
| HP-55/HP-55S | Eosin B | 3.6 | good | Acceptable |
| (1:3) | | | | |
| HP-55S/AS-HF | Eosin B | 3.2 | good | Acceptable |
| (1:1) | | | | |
| HP-55S/AS-HF | Eosin B | 3.3 | good | Excellent |
| (3:1) | | | | |

### Example 7

### Coloured Foam Stability in the Presence of External Fluid Phases

A coloured foam was prepared using the methodology described in example 1 and set in contact with a shower gel suspending base at pH 6.0. Penetration scan experiments were conducted on the system so obtained. These showed that the system was completely stable for several weeks with no migration of dye from the coloured foam into the shower gel suspending base. This demonstrates that the stability of coloured foams according to the invention is not significantly affected despite the presence of an external fluid phase.

However the strong adsorption affinity of the dye for the enteric polymer can be disrupted as the pH of the surrounding medium increases. When the pH of the above system is raised above pH=6.5 the dye is desorbed and diffusional migration starts taking place.

### Example 8

### Perfumed Foam

Hypromellose phthalate (hydroxypropylmethylcellulose phthalate, grade HP-55 ex Shin Etsu Chemical Co., Ltd. (Tokyo, Japan)) was made up as a stock solution (10 w/v % in water, pH 5.6) by mixing 10 g of HP-55 in 70 mL of DI water, followed by the addition of 1N NaOH solution to adjust pH 5.6. This mixture was stirred for 12 hours to obtain homogeneous clear solution, and then final total volume was adjusted to 100 mL by adding DI water.

LH-22 (Low-substituted hydroxypropyl cellulose ex Shin Etsu Chemical Co., Ltd. (Tokyo, Japan)) was made up as a stock solution (5 w/v %, pH>12) by mixing 5 g of LH-22 powder in a NaOH solution ˜90 ml (10 w/v % solution). This solution was stirred using magnetic bar (for 1-2 days) to obtain homogeneous clear solution. When a clear solution was obtained, the final total volume was adjusted to 100 mL by adding NaOH 10% solution.

Cellulose particle stabilized foams were prepared in situ using a high-speed blender (Oster Model 4242, Sunbeam Products, Inc., Boca Raton, Fla.). Pre-mixed solutions of varying amounts of HP-55 or LH-22 stock solution and benzyl acetate (perfume) were slowly poured into the blender running at 15,000 rpm containing DI water where hydrochloric acid was added to adjust the pH of the final foam suspension. The foams formed immediately during the blending process for 60 s and were then transferred into a 250 mL graduated cylinder.

To evaluate quantitatively the volatility of the perfume compounds from the foam sample, we performed a gas chromatograph analysis. As soon as the foam samples (10 mL) were formed, they were placed in air tight vials (20 mL) sealed with a silicon septum, and allowed to age for at least 2 days in room temperature. For the temperature study, the sample vials were allowed to equilibrate in a water bath for 30 min in prior to injection into the gas chromatograph. Approximately 200 μL of vapour above the foam sample was drawn out from the vial with a gas-tight syringe. Then it was injected into the gas chromatograph system (Agilent Technologies 6890N Network GC system) equipped with DB5 column (temperature profile: 100° C. to 235° C. with 20° C./min ramping rate).

The effect of HP-55 amount on BA (benzyl acetate) release is shown in Table 6. The intensity of BA peak in gas chromatograph is gradually decreased as the amount of HP-55 increases. These results indicate that HP-55 particles are very effective for the sustained release of perfumes (i.e. BA). The effect of LH-22 particles is even more pronounced than with HP-55 in suppressing BA release.

TABLE 6

| | HP-55 | LH-22 | BA | Water | | Height of BA |
|---|---|---|---|---|---|---|
| | (g) | (g) | (mL) | (mL) | pH | peak in GC |
| 1 | 0 | | 0.2 | 99.8 | 3.5 | 466,000 |
| 2 | 0.4 | | 0.2 | 99.3 | 3.5 | 428,000 |
| 3 | 1 | | 0.2 | 98.8 | 3.5 | 353,000 |
| 4 | 2 | | 0.2 | 97.8 | 3.5 | 251,000 |
| 5 | 4 | | 0.2 | 95.8 | 3.5 | 171,000 |
| 6 | 6 | | 0.2 | 93.8 | 3.5 | 164,000 |
| 7 | 8 | | 0.2 | 91.8 | 3.5 | 135,000 |
| 8 | | 2 | 0.2 | 97.8 | 3.5 | 65,000 |

The amount of BA perfume release was analyzed at various temperatures (Table 7). In general, the BA release increases as temperature increases at any formulation, due to the increasing vapour pressure of BA. Table 7 shows that the addition of HP-55 particles effectively suppresses the BA release at a given temperature conditions (25-75° C.) as compared to the formulation without HP-55. The addition of only 2% of HP-55 in formulation can suppress 50-70% of BA release at given temperature conditions.

TABLE 7

| | | | | | Height of |
|---|---|---|---|---|---|
| | HP-55 | BA | Water | Temperature | BA peak in |
| | (g) | (mL) | (mL) | (° C.) | GC |
| 1 | | 0.2 | 99.8 | 25 | 466,000 |
| 2 | | 0.2 | 99.8 | 35 | 28,455,000 |
| 3 | | 0.2 | 99.8 | 45 | 59,587,000 |
| 4 | | 0.2 | 99.8 | 55 | 98,522,000 |
| 5 | | 0.2 | 99.8 | 65 | 187,229,000 |
| 6 | | 0.2 | 99.8 | 75 | 345,178,000 |
| 7 | 2 | 0.2 | 97.8 | 25 | 251,000 |
| 8 | 2 | 0.2 | 97.8 | 35 | 6,969,000 |
| 9 | 2 | 0.2 | 97.8 | 45 | 17,638,000 |
| 10 | 2 | 0.2 | 97.8 | 55 | 42,064,000 |
| 11 | 2 | 0.2 | 97.8 | 65 | 58,148,000 |
| 12 | 2 | 0.2 | 97.8 | 75 | 140,216,000 |

### Example 9

### Perfume Accumulation at Water/Oil Interfaces

In order to detect the presence of BA, we prepared an emulsion stabilized with HP-55 containing a dye stained-BA. The dye used was Nile Red (lipophilic fluorescence) dye ex Aldrich. The fluorescence image indicated that the most of BA was localized at the interface of droplet/surrounding media. During the particle formation, BA appears to be incorporated within the HP-55 particles, which are subsequently positioned at the interface of droplet/media.

### Example 10

### Composition Made with Injected Colored and Perfumed Foams

Colored and perfumed foams prepared following the methodologies described above show good mechanical properties and can stay unchanged on their own (i.e., separated from the liquid phase underneath). It is possible to load the foam into a syringe, or other positive displacement device, and subsequently inject the foam into a distinct structured liquid phase exhibiting yield stress. The injection produces visually appealing motives reminiscent of fractal patterns commonly found in nature. The patterns are believed to consist of: colored or perfume foams; free and transparent air bubbles of different sizes; as well as liquid from the wet foam. Without being bound by theory, the formation of such fractal motives is thought to be created by the mismatch in flow rheology between the injected foam and the structured liquid medium. Such visually striking motives will be appealing when incorporated into home and personal care products; foods, etc.

In one of the examples, two colored foams were prepared according to standard procedures described above. Each colored-foam was loaded into a 5 ml plastic syringe and then injected in a sequential fashion into a gel composition. The transparent gel material used was a polyacrylic-based Aqua CC Carbopol gel (Sasol advanced materials), which according to the manufacturer, reaches a yield stress of about 90 Pa and maximum transparency at pH 3.5.

1. A composition comprising at least two immiscible fluid phases separated by a particle modified fluid-fluid interface, in which the interface is stabilised by biopolymeric microparticles adsorbed at the interface, characterised in that the properties of the interface are further modified via the association of at least one functional group on the biopolymer with at least one ligand.

2. A composition according to claim 1, in which the biopolymeric microparticles are anisotropic.

3. A composition according to claim 1, in which the biopolymer used to form the microparticles is a hydrophobically substituted polysaccharide whose solubility is a function of pH and/or temperature and which forms microparticles when precipitated from solution.

4. A composition according to claim 3, in which the biopolymer is an enteric polymer selected from hydroxypropyl methyl cellulose acetate succinate (HPMCAS), hydroxypropyl methyl cellulose phthalate (HPMCP), cellulose acetate phthalate (CAP), cellulose acetate trimellitate (CAT), carboxymethyl ethyl cellulose (CMEC), and mixtures thereof.

5. A composition according to claim 1, in which the ligand is an acidic dye, which will protonate at pH 5.0 or less.

6. A composition according to claim 5, in which the acidic dye is an acidic xanthene dye including hydroxyl and/or carboxyl substituent groups in the dye structure.

7. A composition according to claim 6, in which the acidic xanthene dye contains a fluorone nucleus, which is further substituted at various positions with halogen.

8. A composition according to claim 1 in which the ligand is a perfume.

9. A composition according to claim 1, which is formulated as a home or personal care composition comprising one or more surfactants.

10. A composition according to claim 1, which is dispersed into a suspending base comprising one or more suspending agents.

11. A composition according to claim 1, which comprises coloured foam dispersed in a gel.

12. A process for preparing a composition according to claim 1, in which the biopolymeric microparticles are prepared by a precipitation process in which a solution of biopolymer is precipitated under conditions of high shear wherein the solution is subjected to stirring at greater than 7000 rpm.
